(19) United States
(12) Patent Application Publication
(10) Pub. No.: US 2025/0024854 A1
(43) Pub. Date: Jan. 23, 2025

(54) A METHOD OF PRODUCING A COMPOSITION, AN WATER SOLUBLE EXTRACT COMPOSITION AND A NON-WATER SOLUBLE EXTRACT COMPOSITION, EACH COMPOSITION BEING A PHYCOBILIPROTEIN CONTAINING MICRO-ORGANISM-BASED COMPOSITION

(71) Applicant: FUL FOODS B.V., Amsterdam (NL)

(72) Inventors: Julia STREULI, Amsterdam (NL); Cristina PRAT, Amsterdam (NL); Sara GUAGLIO, Amsterdam (NL)

(21) Appl. No.: 18/710,147

(22) PCT Filed: Nov. 15, 2022
(86) PCT No.: PCT/NL2022/050654

§ 371 Date: May 14, 2024

(30) Foreign Application Priority Data
Nov. 15, 2021 (WO) PCT/NL2021/050701

(51) Int. Cl. A23J 1/00; A23J 3/20; A23L 5/43; A23L 5/46; A23L 29/00; A61K 35/748

(52) U.S. Cl. CPC A23J 1/008; A23J 3/20; A23L 5/43; A23L 5/46; A23L 29/065; A61K 35/748

(57) ABSTRACT

The present invention relates to a method of producing a phycobiliprotein-containing micro-organism-based composition, the method comprising the steps of a) reacting phycobiliprotein-containing micro-organisms, water and a salt for a duration of at least 1 hour to provide an aqueous suspension, b) obtaining an acidic aqueous suspension having a pH between 2 and 7 starting from the aqueous suspension obtained in step a), c) removing the solids from the acidic aqueous suspension to obtain the phycobiliprotein-containing micro-organism-based composition. The invention further relates to an aqueous and a dried phycobiliprotein-containing micro-organism-based water soluble extract composition and a aqueous and a dried phycobiliprotein-containing micro-organism-based non-water soluble extract composition.

### TECHNICAL FIELD

The present invention relates to a method of producing a phycobiliprotein-containing micro-organism-based composition, an aqueous and a dried phycobiliprotein-containing micro-organism-based water soluble extract composition, and an aqueous and a dried phycobiliprotein-containing micro-organism-based non-water soluble extract composition.

### BACKGROUND

The background description includes information that may be useful in understanding the present invention. It is not an admission that any of the information provided herein is prior art or relevant to the presently claimed invention, or that any publication specifically or implicitly referenced is prior art.

Phycobiliprotein-containing micro-organisms have been part of the human diet since thousands of years and have been used as a nutrient-dense food source. Historical records suggest that photosynthetic bacteria, such as Arthrospira platensis were consumed by tribes in Africa and by the Aztecs in Mexico.

During the last decades there has been increasing interest in the commercial production of phycobiliprotein-containing micro-organisms for human consumption and/or as feed for livestock. One of the reasons is that phycobiliprotein-containing micro-organisms have demonstrated potential to meet the population's need for a more sustainable food supply, specifically with respect to protein demand. Additionally, phycobiliprotein-containing micro-organisms produce several bioactive compounds with potential benefits for human health.

Phycobiliprotein-containing micro-organisms as a sustainable source of proteins is a rather new idea and could significantly contribute to meet the population's need for protein, with several advantages over other currently used protein sources. Micro-organism based proteins have low land requirements compared to animal-based proteins and some other plant-based proteins.

Among the microalgae genera largely employed for human consumption are (either as a whole or parts thereof): Chlorella (genus) from the phylum Chlorophyta and class of Terbouxiophydeae; Dunaliella (genus) from the phylum Chlorophyta, class of Chlorophyceae, and family of Dunaliellaceae; and Haematococcus (genus) from the phylum Chlorophyta, class of Chlorophyceae, and family of Haematococcus.

Among the various phycobiliprotein-containing micro-organisms that have been explored for their commercial potential is Arthrospira platensis (generally referred to as Spirulina) a species of free-floating filamentous cyanobacteria, providing high-quality proteins with a well-balanced amino acid profile.

In addition, phycobiliprotein-containing micro-organisms as part of the diet may provide potential benefits for health due to the presence of bioactive compounds in the phycobiliprotein-containing micro-organisms (for example, antioxidative, antihypertensive, immunomodulatory, hepato-protective, and anticoagulant activities have been attributed to phycobiliprotein-containing micro-organisms-derived peptides).

Despite the fact that phycobiliprotein-containing micro-organisms clearly show potential as part of sustainable food solutions, utilization of phycobiliprotein-containing micro-organisms or phycobiliprotein-containing micro-organisms-derived products in food products is limited. This is in part due because of the underdeveloped technologies and processes currently available for phycobiliprotein-containing micro-organisms processing towards high quality and palatable food products.

Phycobiliprotein-containing micro-organisms have, for example been incorporated in such products as cookies, biscuits, bread and pasta as these products allow for reasonable acceptance of taste, texture, and appearance, but most phycobiliprotein-containing micro-organisms is still presented in the form of food supplements, powders and tablets.

CN113278528A discloses a method for rapid breaking of the cell walls of Spirulina to extract natural products, the method using a buffer of polycarboxylic acids and salts thereof in a specific ratio. Said buffer is used to ensure that the pH value is kept constant throughout the process, specifically to a value between of pH 6 to 7.5.

CN110903384A discloses an extraction method of the phycobiliprotein phycocyanin using a salt at neutral pH. EP2484230B1 relates to compositions comprising Spirulina-extract for food purposes.

The incorporation of phycobiliprotein-containing micro-organisms into traditional products has until now been found inconvenient because of its colour, its fishy taste, and its strong odour, as well as its powdery consistency and appearance, all adversely affecting consumers' perception about taste and quality. All these aspects constitute main areas for improvement. For example, it has not yet been possible to sufficiently disguise the fishy taste and odour of phycobiliprotein-containing micro-organisms, therefore, limiting the amount to be used in products.

Another issue is the fact that freshly harvested phycobiliprotein-containing micro-organisms can only be preserved for up to a few days. Phycobiliprotein-containing micro-organisms are therefore commercialized, mostly, as dried powders to facilitate their use as food ingredients and to allow easy transportation and long-term stability. The drying process of course increases energy need, thereby reducing the overall sustainability of the product. In addition, these dried or dehydrated phycobiliprotein-containing micro-organisms powders are characterized by loss of nutritional value and a fishy smell.

Another issue is the fact that current phycobiliprotein-containing micro-organisms, specifically phycocyanin-containing compositions, exhibit low stability at low pH (e.g. at a pH below 5 or even below 4 or even below 3.5) and/or at high temperature above 40° C., or above 70° C. or above 90° C. or even 95° C.; making phycobiliprotein-containing compositions very difficult to be integrated in final consumer applications.

In light of this, new phycobiliprotein-containing micro-organisms-based food products, compositions, methods and uses would be highly desirable. In particular, there is a clear need in the art for sustainable phycobiliprotein-containing micro-organisms-based products and methods for producing such products, wherein the products meet nowadays standards of sustainability, taste, odour and nutritional value and quality.

Accordingly, the technical problem underlying the present invention can be seen in the provision of such products, compositions, and methods for complying with any of the aforementioned needs. The technical problem is solved by the embodiments characterized in the claims and herein below.

### SUMMARY

It is an object of the present invention to provide an improved method for preparing phycobiliprotein-containing micro-organisms-based compositions, in particular edible compositions.

It is a further object of the present invention to provide (edible/drinkable) compositions comprising phycobiliprotein-containing micro-organisms.

It is a further a specific object of the present invention to provide phycobiliprotein-containing compositions that are stable at low pH, preferably at a pH below 5, or even below 4 or even below 3.5 and temperature above 40° C., preferably above 70° C., more preferably above 90° C. or even above 95° C.

It is a furthermore a specific object of the present invention to obtain a chlorophyll-containing composition in which the chlorophyll present in the composition is stable in suspension.

In a first aspect, the present invention relates to a method of producing a phycobiliprotein-containing micro-organism-based composition, the method comprising the steps of:
a) reacting phycobiliprotein-containing micro-organisms, water and a salt for a duration of at least 1 hour to provide an aqueous suspension;
b) obtaining an acidic aqueous suspension having a pH between 2 and 7 starting from the aqueous suspension obtained in step a);
c) removing the solids from the acidic aqueous suspension to obtain the phycobiliprotein-containing micro-organism-based composition.

In second aspect, the present invention relates to an aqueous phycobiliprotein-containing (micro-organism-based) water soluble extract composition, comprising:
between 40 and 98 wt. % of water;
between 0.5 and 10 wt. % of an acidifier, preferably selected from the group consisting of citric acid, lactic acid, malic acid, lemon juice or lime juice;
at most 0.5 wt. % of chlorophyll, preferably at most 0.001 wt. %; and
at least 0.5 wt. % of phycocyanin, preferably at least 1 wt. %, more preferably at least 10 wt. %, most preferably at least 25 wt. %;
preferably less than 0.1 wt. %, more preferably less than 0.01 wt. % of a sugar,
preferably at least 0.03 wt. %, more preferably at least 0.1 wt. %. of an alkaline or alkaline earth metal salt cation, preferably a calcium cation; and

wherein the composition preferably has:
a colour value according to the CIELAB colour scale for a of between −10 and −4 and for b of between 10 and −40; and/or
a pH of between 2 and 5.

In a third aspect, the present invention relates to a dried phycobiliprotein-containing (micro-organism-based) water soluble extract composition, comprising:
between 20 and 60 wt. % of proteins;
between 1 and 20 wt. % of carbohydrates;
between 1 and 50 wt. % of one or more acids;
between 0.5 and 10 wt. % of fat;
at most 1 wt. % of chlorophyll, preferably at most 0.1 wt. %; and
at least 5 wt. % of phycocyanin, such as at least 25 wt. % of phycocyanin, preferably at least 40 wt. %, more preferably at least 50 wt. %;
preferably at least 1.5 wt. %, more preferably at most 5 wt. % of an alkaline or alkaline earth metal salt cation, preferably a calcium cation;
preferably less than 1 wt. %, more preferably less than 0.1 wt. % of a sugar; and

wherein the composition preferably has a pH of between 2 and 5.

In a fourth aspect, the present invention relates to an aqueous phycobiliprotein-containing (micro-organism-based) non-water soluble extract composition comprising:
between 50 and 98 wt. % of water;
between 1 and 50 wt. % of protein;
between 1 and 50 wt. % of an acidifier, preferably citric acid, lactic acid, malic acid, lemon juice or lime juice;
at least 0.01 wt. % of chlorophyll, such as at least 0.5 wt. %, preferably at least 1 wt. %, such as at least 2 wt. %;
at least 0.1 wt. % of phycocyanin;
preferably at least 0.03 wt. %, more preferably at least 0.1 wt. % of an alkaline or alkaline earth metal salt cation, preferably a calcium cation; and

wherein the composition preferably has a pH of between 2 and 5.

In a fifth aspect, the present invention relates to a dried phycobiliprotein-containing (micro-organism-based) non-water soluble extract composition comprising:
between 10 and 90 wt. % of proteins;
between 1 and 40 wt. % of carbohydrates;
between 1 and 50 wt. % of an acid
between 1 and 40 wt. % of fat;
at least 1 wt. % of chlorophyll, preferably at least 2 wt. %; such as at least 4 wt. %;
at most 10 wt. % of phycocyanin, preferably at most 0.1 wt. %; and
preferably at least 1.5 wt. %, more preferably at least 5 wt. %. of an alkaline or alkaline earth metal salt cation, preferably a calcium cation; and

wherein the composition preferably has a pH of between 2 and 5.

It is contemplated that any method or composition described herein can be implemented with respect to any other method or composition described herein. Embodiments discussed in the context of methods and/or compositions of the invention may be employed with respect to any other method or composition described herein. Thus, an embodiment pertaining to one method or composition may be applied to other methods and compositions of the invention as well.

### List of Definitions

Various terms relating to the methods, compositions, uses and other aspects of the present invention are used throughout the specification and claims. Such terms are to be given their ordinary meaning in the art to which the invention pertains, unless otherwise indicated.

Other specifically defined terms are to be construed in a manner consistent with the definition provided herein. Although any methods and materials similar or equivalent to those described herein can be used in the practice for testing of the present invention, the preferred materials and methods are described herein.

For purposes of the present invention, the following terms are defined below.

As used herein, the singular form terms “a,” “an,” and “the” include plural referents unless the content clearly dictates otherwise. Thus, for example, reference to “a bacterium” includes a combination of two or more individual bacteria, and the like.

As used herein, “and/or” refers to a situation wherein one or more of the stated cases may occur, alone or in combination with at least one of the stated cases, up to with all of the stated cases.

As used herein, “at least” a particular value means that particular value or more. For example, “at least 2” is understood to be the same as “2 or more” i.e., 2, 3, 4, 5, 6, 7, 8, 9, 10, 11, 12, 13, 14, 15, etc. As used herein, the term “at most” a particular value means that particular value or less. For example, “at most 5” is understood to be the same as “5 or less” i.e., 5, 4, 3, −10, −11, etc.

As used herein, “comprising” or “to comprise” is construed as being inclusive and open ended, and not exclusive. Specifically, the terms and variations of the specified features, steps or components are included. These terms are not to be interpreted to exclude the presence of other features, steps or components. It also encompasses the more limiting “to consist of”.

As used herein, “as is known to the skilled person” refers to a situation wherein the methods of carrying out the conventional techniques used in methods of the invention will be evident to the skilled worker. The practice of conventional techniques in molecular biology, biochemistry, cell culture, genomics, sequencing, medical treatment, pharmacology, immunology and related fields are well-known to those of skill in the art and are discussed, in various handbooks and literature references.

As used herein, “micro-organism” refers to a microscopic organism, which may exist in its single-celled form or in the form of a colony of cells. Micro-organism include all unicellular organisms. All of the Archaea and Bacteria are micro-organisms; since they lack cell nuclei they are prokaryotes. In addition, micro-algae, such as red algae, are micro-organisms; since they have a nucleus enclosed with a nuclear envelop they are eukaryotes.

As used herein, “phycobiliprotein-containing micro-organism” refers to photoautotrophic micro-organisms that comprise light harvesting complexes (phycobilisomes) comprising phycobiliproteins; examples of such complexes are chloroplasts comprising chlorophyll, phycocyanin, and/or phycoerythrin. Chlorophyll is the name for a group of green pigments, present in e.g. green algae, allowing phycobiliprotein-containing micro-organisms to absorb energy from light. Phycocyanin is a blue protein-pigment complex, present in e.g. cyanobacteria, which are accessory to chlorophyll. Phycoerythrin is a red protein-pigment complex, present in e.g. red algae and cyanobacteria, which are accessory to chlorophyll pigments. Photoautotrophic micro-organisms are able to convert light energy, carbon dioxide and water into chemical energy stored in carbohydrate molecules, such as sugars and starches. The phycobilisome of cyanobacteria comprise phycocyanin, phycoerythrin and chlorophyll. The phycobilisome of red algae comprise phycoerythrin and chlorophyl. Phycocyanin and phycoerythrin are pigment-protein complexes that are accessory to the pigment chlorophyl.

As used herein, “microalgae” refers to a microscopic algae, typically found in freshwater and marine systems, living in both the water column and sediment. Micro-algae are multi- and unicellular organisms existing individually or in chains or groups. Depending on the species, the size can range from a few to a few hundred micrometres.

As used herein, “Rhodophyta” refers to a phylum of red algae. Rhodophyta, which are mainly marine, are multi- and unicellular, and cell wall components are cellulose and sulphated phycocolloids. The main photosynthetic pigments are chlorophyll a, chlorophyll d, and phycoerythrin.

As used herein, “Cyanophyta”, also known as cyanobacteria, and blue-green bacteria, are a phylum of Gram-negative bacteria that obtain energy via photosynthesis. Cyanobacteria are also known as blue-green algae, although the term algae is restricted to eukaryotes and do not apply to cyanobacteria, which are prokaryotes.

As used herein, “Arthrospira” is a genus of free-floating filamentous cyanobacteria (oxygenic photosynthetic) characterized by cylindrical, multicellular trichomes (outgrowths) in an open left-hand helix. As used herein, “Arthrospira maxima”, refers to the species Arthrospira maxima of the genus Arthrospira, “Arthrospira platensis” (also known as Arthrospira spirulina platensis), refers to the species Arthrospira platensis of the genus Arthrospira, and “Arthrospira fusiformis”, refers to the species Arthrospira fusiformis of the genus Arthrospira.

As used herein, “fermentation”, “ferment” or “lactic acid fermentation” refers to a type of anaerobic fermentation by which sugars are converted into cellular energy and lactate (lactic acid in solution).

As used herein, “lactic acid producing bacterium”, refers to any bacterium that mainly or predominantly produces lactic acid during fermentation. A homofermentative lactic acid producing bacterium converts sugars to lactic acids as the only or major end-product, while a heterofermentative lactic acid producing bacterium may produce lactic acid and additional metabolites such as ethanol, acetic acid and carbon dioxide. As used herein, “Lactobacilli”, refers to bacteria of the genus Lactobacillus of Gram-positive, facultative anaerobic, rod-shaped, non-spore-forming bacteria. They are a major part of lactic acid producing bacteria, converting sugars into lactic acid. As used herein, “Lactobacillus plantarum”, refers to species Lactobacillus plantarum of the genus Lactobacillus of Gram-positive, facultative anaerobic, rod-shaped, non-spore-forming bacteria. They are a major part of lactic acid producing bacteria, converting sugars into lactic acid. As used herein, “Bifidobacteria”, refers to the major genera of bacteria that make up the gastrointestinal tract microbiota in mammals, including humans. Some Bifidobacteria are used as probiotics. The genus Bifidobacterium refers to the genus of gram-positive, non-motile, often branched anaerobic bacteria.

As used herein, “fresh phycobiliprotein-containing micro-organisms”, refers to phycobiliprotein-containing micro-organisms that have not been dried or dehydrated. The term is intended to mean a non-dried, non-dehydrated phycobiliprotein-containing micro-organisms harvested less than 48 hours previously, but also includes phycobiliprotein-containing micro-organisms that after being harvested are immediately cooled or frozen, without drying or dehydration.

As used herein, “phycobiliprotein-containing micro-organisms-based composition” refers to a composition according to the present invention that is prepared using phycobiliprotein-containing micro-organisms and that contains one or more components of said phycobiliprotein-containing micro-organisms.

As used herein, “fatty acids” refers to free fatty acids (FFAs), monoacyl glycerides (MAGs), diacyl glycerides (DAGs), and triacyl glycerides (TAGs) or one or more combinations thereof.

As used herein, “solids”, also called “non-water soluble extract”, refers to the solid substances in the aqueous suspension obtained through the method of the invention, and as mentioned in step c of claim 1.

As used herein, “sugar”, refers to a simple sugars being monosaccharides and disaccharides. Examples are glucose, fructose and galactose, lactose, sucrose and mannose.

### BRIEF DESCRIPTION OF DRAWINGS

The present invention is described hereinafter with reference to the accompanying drawings in which embodiments of the present invention are shown and in which like reference numbers indicate the same or similar elements.

FIG. 1 shows a graph showing the effect of the amount of salt (CaCl₂) on the amount of recovered pigments (phycocyanin, phycoerythrin, and chlorophyll).

FIG. 2 shows the results of measurements of phycocyanin content (in mg/ml) in relation to reaction duration of step a) of the present method.

FIG. 3A shows a graph and FIGS. 3B and 3C show pictures, all showing the effect of using a salt versus using a buffer during step a of claim 1.

FIG. 4 shows a graph showing the effect of using an acidic pH during step b of claim 1.

FIGS. 5A, 5B, 5C, and 5D show the colour stability of the present composition at low levels of pH after pasteurization at elevated temperature.

FIG. 6 shows a graph showing the transmittance peak factors for compositions according to the present invention.

### DESCRIPTION OF EMBODIMENTS

The aspects of the present invention will be discussed in more detail below.

The present invention relates is a first aspect to a method of producing a phycobiliprotein-containing micro-organism-based composition, said method comprising three steps. The three steps being in order: a) reacting phycobiliprotein-containing micro-organisms, water and a salt for a duration of at least 1 hour to provide an aqueous suspension; b) obtaining an acidic aqueous suspension having a pH between 2 and 7 starting from the aqueous suspension obtained in step a); c) removing the solids from the acidic aqueous suspension to obtain the phycobiliprotein-containing micro-organism-based composition. The different features of the present invention will be discussed in more detail below.

### Step A) Aqueous Suspension of Phycobiliprotein-Containing Micro-Organisms

In step a) a mixture is prepared of phycobiliprotein-containing micro-organisms, water and a salt. This mixture is allowed to react for a duration of at least 1 hour. This provides an aqueous suspension. During step a), the presence of the salt and water will lead to osmotic shock of the phycobiliprotein-containing micro-organisms. This osmotic shock will lead to cell disruption and nutrient solubilization.

### Phycobiliprotein-Containing Micro-Organisms

In step a) an aqueous suspension is prepared or obtained comprising at least one species of a phycobiliprotein-containing micro-organism.

In an embodiment, the phycobiliprotein-containing micro-organisms are present in the aqueous suspension in an amount of between 30 and 60 weight %, based on the weight of the aqueous suspension.

The phycobiliprotein-containing micro-organisms used in preparing the aqueous suspension may be any phycobiliprotein-containing micro-organism that may be suitable to be included in a food product, i.e. which can safely be consumed by a person, and at least in the amounts provided in the end product. The phycobiliprotein-containing micro-organisms may be provided as dried phycobiliprotein-containing micro-organisms; most of the phycobiliprotein-containing micro-organisms present as raw material in the market are available as dried or dehydrated phycobiliprotein-containing micro-organism, in particular in the form of powder.

In preferred embodiments of the method according to the invention, the at least one phycobiliprotein-containing micro-organism is selected from the group consisting of Cyanophyta (cyanobacteria), such as Arthrospira spp., like Arthrospira platensis, Arthrospira maxima, and Arthrospira fusiformis, and Rhodophyta (red algae), and combinations thereof.

Use of phycobiliprotein-containing micro-organisms, in particular the aforementioned species, result in a composition with a high nutritional profile, that is, having a high protein content, including containing all nine essential amino acids, being high in antioxidants (minerals and trace elements), containing omega-type fatty acids, being high in fibre, being hypolipidemic (lowering lipid concentration in blood), and exhibiting anti-obesity effects.

As described herein, it is contemplated that in some embodiments according to the invention, only one type (species) of phycobiliprotein-containing micro-organisms is used. In some embodiments more than one type (species) of phycobiliprotein-containing micro-organisms, i.e. a combination of phycobiliprotein-containing micro-organisms is used. For example, a combination of one or more Arthrospira spp., and one or more Galdieria spp., and in any suitable ratio, may be used.

In a preferred embodiment only Arthrospira spp. are used, for example Arthrospira platensis, Arthrospira maxima, or a combination thereof, in the method and composition according to the invention.

Further, and importantly, in a preferred embodiment, the phycobiliprotein-containing micro-organisms used in the aqueous suspension are fresh phycobiliprotein-containing micro-organisms, i.e. phycobiliprotein-containing micro-organisms that have not been extensively dried after harvesting. Preferably, fresh Arthrospira spp., such as Arthrospira platensis, Arthrospira maxima, and Galdieria spp., such as Galdieria sulphuraria and combinations thereof is used, most preferably fresh Arthrospira platensis.

Normally, fresh phycobiliprotein-containing micro-organisms are dried within 48 hours, 24 hours or even less than 12 hours after harvesting, depending on the type of phycobiliprotein-containing micro-organisms. The fresh phycobiliprotein-containing micro-organisms may after being harvested also be cooled or frozen and used in the method of the invention. It was found that although dried phycobiliprotein-containing micro-organisms may be used in the method according to the invention, and therefore in the composition of the invention, fresh phycobiliprotein-containing micro-organisms provide for a better quality product. Better quality is for example seen in enhanced digestibility, a high nutritional profile, and increased bio-assimilation, higher palatability and increased stability. Despite the fact that the use of a fresh form of phycobiliprotein-containing micro-organisms is not widespread owing to the fact that it rapidly deteriorates shortly after harvesting, it was found that in the context of the current invention fresh phycobiliprotein-containing micro-organisms are preferred. An embodiment, a combination of both fresh and dried phycobiliprotein-containing micro-organisms may be used.

In a preferred embodiment, the present invention relates to a method of producing a cyanobacteria based composition, the method comprising the steps of: a) reacting cyanobacteria, water and a salt for a duration of at least 1 hour to provide an aqueous suspension; b) obtaining an acidic aqueous suspension having a pH between 2 and 7 starting from the aqueous suspension obtained in step a); c) removing the solids from the acidic aqueous suspension to obtain the cyanobacteria-based composition.

### Water

In step a) an aqueous suspension is prepared. Water forms a large part of this suspension. The water may be selected from the group consisting of mineral water, demineralized water, deosmotized water, deionized water and distilled water. When demineralized water is used, the osmotic shock observed during step a is higher than when non-demineralized water is used.

Tap water may contain chlorine, chloramine, fluoride and/or other contaminants, detrimental to the lactic acid producing bacteria when fermentation is used (see step b) below). Demineralized, deionized and distilled water don't contain these contaminants, thus contributing to the thriving of the lacto-fermenting bacteria, and thus contributing to a high nutritional profile end product.

Although not in particular limited to a specific temperature, it was found that the temperature of the water used for preparing the aqueous suspension may be of a temperature of between 1-25° C.

### Salt

In step a) an aqueous suspension is prepared or obtained comprising a salt. This salt is an additional source of salt, i.e. a (source of) salt other than the salts that may naturally be present in the phycobilin-protein-containing micro-organisms.

In an embodiment, salt is present having an osmolarity of between 2 and 140 mOsmol/l. In an embodiment, salt is present in an amount of between 0.1 and 5.0% (w/w), preferably between 0.5 and 3.0% (w/w), preferably between 1.0 and 2.5% (w/w), based on the weight of the phycobiliprotein-containing micro-organism.

The present inventors have observed that the use of a salt provides good results in view of osmotic shock, better than when instead of salt only heating is used. In the present invention the salt should be a food safe salt. In a preferred embodiment of the method according to the invention, an alkaline or alkaline earth metal salt, preferably a sodium, potassium, calcium or magnesium salt, more preferably the chloride salt thereof. Without wishing to be bound by a particular theory, the present inventors have observed that the positive charged alkaline (earth) metal ion has a positive effect on the colour stability of the composition obtained by the method of the invention.

As discussed above CN113278528A uses a specific buffer during the extraction process to keep the pH constantly between 6 and 7.5. To test the effect of the use of a salt according to the present invention versus the use of a carboxylic buffer (comprising a salt) not according to the present invention experiments were carried out and these are discussed in Example 3 below. The results very clearly show that when a buffer is used in step a) instead of a salt at a temperature of 10° C., the extraction of phycobiliproteins is much less efficient.

### Preparation of Suspension and Amounts

The aqueous suspension may be prepared by first dissolving the salt in the water and then adding the phycobiliprotein-containing micro-organisms.

Although not in particular limited, preferably the aqueous suspension is prepared in or provided to a sterile or non-sterile mashing tank.

In an embodiment, the aqueous suspension obtained in step a) comprises between 35 and 69.9 wt. % of water, between 30 and 60 wt. % of phycobiliprotein-containing micro-organisms (viz. in the form of a fresh paste), and between 0.1 and 5.0 wt. % salt, based on the weight of the aqueous suspension, wherein the combined amount of water, phycobiliprotein-containing micro-organisms and salt is 100 wt. %. The amount of phycobiliprotein-containing micro-organism solids in step a) is preferably between 0.5 wt. % and 50 wt. %, preferably between 1 wt. % and 20 wt. %, preferably between 2 wt. % and 10 wt. %, such as between 5-10 wt. % (equivalent to 25-40 wt. % of fresh paste). These amounts of solids are calculated from the phycobiliprotein-containing micro-organisms used and do not include the amount of salt that is added.

### Temperature and Duration of Step a)

Step a) is carried out for a duration of at least 1 hour. However, it is preferred that the step is carried out during at least 5 hours, preferably at least 8 hours, preferably at least 10 hours, more preferably at least 20 hours. It is known to a person skilled in the art that with an increase in the vessel size, there will be an increase required in the duration to achieve a same extraction level. The pH during step a) may be pH>5 (above 5), preferably between pH 5 and 6, such as between pH 5.5 and 6.

In an embodiment, step a) (and/or step b) and/or step c)) is/are carried out at a temperature of at most 25° C., preferably at most 20° C., preferably at most 15° C., preferably at most 10° C., preferably at most 5° C. Lower temperatures will reduce the growth rate any possible contamination and will lead to less bacterial growth. It is known to a person skilled in the art that with a decrease in the temperature, there will be an increase required in the duration to achieve the same extraction level.

In an embodiment, the temperature during step a) is at most 5° C. and the pH is >5, preferably with a duration of at least 10 hours.

### Additional Ingredients

It will be understood by the skilled person that all kinds of sweeteners, flavours, antioxidants, emulsifiers, preservatives, colourants, gellants and any combination thereof, may also be added. No added sugar is required for the present method, this is advantageous in case low-carb compositions are desired. As will be disclosed below under step b), in certain embodiments for which fermentation is used, sugar can be added during step a).

### Step B) Acidic Aqueous Suspension

In step b) an acidic aqueous suspension is obtained starting from the aqueous suspension of step a). The pH of acidic aqueous suspension should be between pH 2 and pH 7, such as at most pH 6 or pH 5. Preferably, the pH in step b) is between 2 and 5. The preferred pH is between pH 3 and pH 4.5. It was found that with pH values between these limits the best compositions were obtained by the method of the invention. The use of an acid pH leads to improved colour stabilization as shown by the Examples. CN110903384A discloses an extraction method of phycocyanin using a salt but this does not lead to a colour that is stable at low pH values. Example 4 shows the effect of adding an acidifier in step b) of the process compared to not adding an acidifier in step b) of the process. This example clearly shows that the process according to the present invention, being a combination of first a salt-assisted cell wall breakage, followed by lowering the pH in step b) has a positive effect on the colour stability. Example 5A shows the effect of heat treatment on a sample in which in step b) and acidic pH is used and a comparative.

In step b) the acidic aqueous suspension may be obtained by: i) adding an acidifier, ii) lactic acid fermentation; or iii) a combination thereof. The acidity of the aqueous suspension may increase naturally due to the activity of the lactic acid producing bacteria (ii), but can also be increased by adding an acidifier (i). Theoretically, the acidic aqueous suspension may also be obtained by a process, called “wild” fermentation, referring to fermentation through indigenous bacteria, yeasts and moulds, occurring naturally on the micro-organisms, instead of inoculation of the suspension with lactic acid producing bacteria. However, this is not part of the present invention since wild fermentation was found by the present inventors to take a very long time (weeks) and have additional drawbacks, such as the growth of disease-causing bacteria during the fermentation process. These drawbacks are not observed by the addition of an acidifier and/or the fermentation according to the present invention.

Increase of acidity increases the shelf life of the composition by inhibiting or slowing down the action of enzymes producing the chemical changes in the composition, and preventing the overgrowth of spoilage and disease-causing bacteria, yeasts and moulds, since these microbes need an alkaline environment.

In an embodiment, additional water may be added to the suspension during step b) to further dilute the suspension. Under a) types of water that can be used are disclosed. These are also suitable for addition in step b).

### Acidifier

In order to obtain an acidic aqueous suspension an acidifier may be used. In an embodiment, step b) comprises adding to the aqueous suspension of step a) an acidifier, preferably selected from the group consisting of citric acid, lactic acid, malic acid, lemon juice, lime juice and a combination of two or more thereof, such as citric acid.

The specific amount in weight % of acidifier as such is not relevant, it is added to achieve the desired pH value and the amount needed will depend on the acidity of the acidifier selected. In some embodiments the pH value is set by using strong or weak acids and/or bases. In some embodiments, the acids and/or bases are provided to the aqueous suspension as pure compounds. In some embodiments natural sources, for example juices comprising acids such as lemon or lime juice are used to set the pH value of the aqueous suspension.

When using an acidifier instead of fermentation by lactic acid bacteria, the present inventors observed the final taste of the composition to be more pleasant and and/or the colour value to be improved.

### Fermentation

Instead of or in addition to the addition of an acidifier in step b), fermentation can be used. Lactic acid-fermentation according to the present invention using phycobiliprotein-containing micro-organisms as the (preferably sole) substrate for lactic acid producing bacteria results in excellent compositions. After the fermentation a composition is obtained wherein the composition is characterized by digestibility, high nutritional profile, and bio-assimilation, as well as having a well-accepted taste, odour, and texture and is perceived as highly palatable.

The pH value during fermentation may be monitored. It may for example be decided to stop fermentation at a certain pH value or after a certain amount of time. Alternatively pH may be monitored and adjusted during fermentation.

In some embodiments fermentation is stopped when most of the carbohydrate content of the phycobiliprotein-containing microorganisms that were added to the aqueous suspension has been consumed, for example when 50%, 60%, 70%, 80%, 90%, 95% of the carbohydrate content initially provided in the aqueous suspension has been consumed during fermentation. As will be understood by the skilled person, allowing large part of the carbohydrate content to be fermented in this step can provide for a composition that is characterized by low carbohydrate content, or can even be marketed as carbohydrate-free. Preferably, the fermentation is performed at a pH and at a temperature that allows good to optimal metabolic activity of the fermenting bacteria.

In preferred embodiments, further ingredients are provided to the fermented aqueous suspension, preferably an acidifier. Increase of acidity increases the shelf life of the composition by inhibiting or slowing down the action of enzymes producing the chemical changes in the composition, and preventing the overgrowth of spoilage and disease-causing bacteria, yeasts and moulds, since these microbes thrive in an alkaline environment.

### Lactic Acid Producing Bacteria

The lactic acid producing bacteria may be any kind or strain of bacteria that may safely be used in the preparation of food, and feed products, cosmetics and skin care products. The skilled person is well aware of the various lactic acid producing bacteria that are available to him for use in the method of the invention. The lactic acid producing bacteria may be provided to the aqueous suspension by any means known to the skilled person. In some (preferred) embodiments the lactic acid producing bacterium is provided in the form of a starter culture (or fermentation starter—comprising metabolic active bacteria in a suitable medium) and may already be metabolic active. In other embodiments, the lactic acid bacterium is provided as freeze dried material. Preferably, one type of lactic acid producing bacterium is used, although in some embodiment a combination of different lactic acid bacteria may be used. The skilled person is well aware of suitable conditions to allow fermentation of the inoculated aqueous suspension, for example with respect to temperature, pH (control), stirring, oxygen tension and duration. For example, during fermentation pH, temperature and the like may be monitored and if so desired adjusted in order to allow for optimal fermentation of the inoculated aqueous suspension comprising the at phycobiliprotein-containing micro-organisms. The fermentation is performed by added, not indigenous, lactic acid bacteria.

In a preferred embodiment the type or strain of lactic acid bacteria is selected from Bifidobacteria and/or Lactobacilli, more preferably wherein the bacterium is Lactobacillus plantarum.

The inventors performed tests with a wide variety of bacteria, including single species as well as combination of different species, and although acceptable results were obtained with various different types, best results were obtained with lactic acid producing bacteria, in particular, Bifidobacteria and/or Lactobacilli, including various Lactobacillus species such as Lactobacillus delbruecki, Lactobacillus brevis, Lactobacillus helveticus, and Lactobacillus plantarum.

In preferred embodiments between 0.1×10¹⁰ and 5×10¹⁰ CFU/L, preferably between 1×10¹⁰ and 3×10¹⁰ CFU/L of the bacterium is inoculated. Although the skilled person understand that different amounts (CFU) may be used, it was found that the combination of the phycobiliprotein-containing micro-organisms, an amount of sugar in the aqueous suspension, if any, and the preferred amount of bacteria, provide a very good results with respect to the end product, the composition according to the invention.

High salt tolerance gives lactic acid bacteria an advantage over other less tolerant species and allows the lactic acid bacteria to start fermenting, which produces acid, which further inhibits the growth of non-desirable organisms. It should be noted that salts comprising anti-caking agents and/or iodine might inhibits the fermenting process.

### Additional Ingredients

It will be understood by the skilled person that all kinds of sweeteners, flavours, antioxidants, emulsifiers, preservatives, colourants, gellants and any combination thereof, may also be added.

Sugar may be present in the present invention, although not preferred in most embodiments. The lactic acid producing bacteria use the phycobiliprotein-containing micro-organisms present in the aqueous suspension as sole substrate, while producing lactic acid and thus fermenting the phycobiliprotein-containing micro-organisms present in the aqueous suspension, so no sugar is required. However, the skilled person will understand that too low carbohydrate content will hamper efficient fermentation, whereas too high levels of sugar may likewise not be beneficial for an efficient fermentation, for example because only a small part of total sugar may be consumed during the period of fermentation, lactic acid producing bacteria may become too active, or too much lactic acid producing bacteria may end up in the product, and/or the product becomes too acidic. Hence, in certain embodiments, sugar may be added to aqueous suspension of step a). After having performed various test with different concentrations of sugars it was found that to the aqueous suspension may be added between 0.5-4 wt. % sugar before the fermentation is started, preferably between 1.0 and 2.5 wt. % sugar. Preferably the sugar is selected from the group consisting of monosaccharides and disaccharides, in particular glucose, fructose, galactose, lactose, sucrose, and mannose and combinations thereof. The sugars may be provided as pure compounds, alone or in combination, and/or may be provided as part of a composition, for example as comprised in particular fruit juices such as apple juice (comprising, for example glucose, fructose, and sucrose).

In an embodiment, during step a) as most 0.5 wt. % of a sugar is added, preferably less than 0.1 wt. %, more preferably no sugar is added. In an embodiment, during the complete process as most 0.5 wt. % of a sugar is added, preferably less than 0.1 wt. %, more preferably no sugar is added.

### Temperature and Duration of Step b) and Composition of Suspension

In an embodiment, the temperature during step b) in case of fermentation is between 25-40° C., preferably between 32-38° C. The duration of step b) in case of fermentation may be between 2-72 hours, preferably between 12-48 hours, in order to achieve sufficient fermentation to allow sufficient acid to form. It was found that within this range good quality end products, according to the invention are obtained. It was for example found when fermentation is used that it is possible to obtain end products with very low carbohydrate content, indication that almost all carbohydrates and, added sugars, if any, originally present in the aqueous suspension before fermentation have been consumed by the lactic acid producing bacteria, and such products were found to have very good taste, smell and texture.

The duration of step b) with acidification may be the same as the duration of adding the acidifier, or the composition may be allowed to react after acidification. In an embodiment, the temperature during step b) in case of adding an acidifier is between 0-10° C., preferably between 0-5° C. The duration of step b) in case of adding an acidifier is not limited but may be between 1 and 20 minutes.

In an embodiment, flocculant is added in an amount of between 0.01% and 1 wt. % based on the total weight of the aqueous suspension to improve separation of the soluble fraction. A person skilled in the art will be aware of types of flocculants that may be used.

### Step C) the Phycobiliprotein-Containing Micro-Organism-Based Composition

In step c) the solids are removed from the acidic aqueous suspension to obtain the phycobiliprotein-containing micro-organism-based composition.

In an embodiment, step c) comprises separating the acidic aqueous suspension obtained in step b) into an aqueous phycobiliprotein-containing micro-organism-based water soluble extract composition and a phycobiliprotein-containing micro-organism-based non-water soluble extract composition.

Both the water soluble extract composition as the non-water soluble extract composition have a high nutritional/nourishment profile, are well digestible and absorbable and are high in probiotics, and thus both may be used, neither one is considered waste. The water soluble extract composition (either as such or concentrated/dried), may be used as a natural, safe and healthy additive to food, feed, cosmetics and skin care products. The non-water soluble extract composition (either as such or concentrated/dried), may be used as an enriching additive as well, in particular as a natural and healthy filler to bulk up food and feed.

### Removal of Solids

Removal of the solids may be achieved through centrifugation or filtration, optionally preceded by static sedimentation or decantation. In an embodiment, in step c) is carried out using centrifugation, preferably at a G-force between 2000 and 20000 Relative Centrifugal Force (RCF). Centrifugation as another means to separate the molecules having different densities in the acidic aqueous suspension, allows also to put to use each of the different phases appropriately.

### Temperature and Duration of Step c)

The temperature and duration of step c) depend on the type of removal of solids used and can be determined by a person skilled in the art. In an embodiment, step c) is carried out at a temperature of at most 25° C., preferably at most 20° C., preferably at most 15° C., preferably at most 10° C., preferably at most 5° C.

### Temperature of Full Process—Specific Embodiment

In an embodiment, all steps of the method are carried out at a temperature of at most 10° C., preferably at most 5° C.

### Additional Steps

One or more additional steps may be carried out prior to and or after the steps a), b) and c) cited above. Several of these additional steps are disclosed in detail below. These are:
step d) of washing
step e) of pasteurizing
step f) of concentrating or drying
step g) of purifying

In a specific embodiment, the present method comprises sequential steps of a, b, c and the following additional steps: d; d+e; d+e+f; d+e+f+g; d+f; d+f+g; d+g; e; e+f; e+f+g; f; f+g; or g.

### Step d) of Washing

In an embodiment, the method comprises an additional step d) of washing the solids removed in step c) to obtain additional composition comprising phycobiliprotein-containing micro-organisms that is combined with the composition comprising phycobiliprotein-containing micro-organisms obtained in step c). This will increase the yield.

It will be understood by the skilled person that all features, preferences and characteristics already presented herein within the context of the method according to the invention likewise applies to the combination of the washed solids and the phycobiliprotein-containing micro-organisms composition obtained in step c, according to the invention, as well as the water soluble extract composition, dried water soluble extract composition, non-water soluble extract composition, dried non-water soluble extract composition, pasteurized and purified composition obtained through the method according to the invention, and such features, preferences and characteristics will therefore not be repeated below.

In an embodiment, the temperature during this step is between 1 and 10° C. In an embodiment, the duration of this step is between 1 minute and 2 hours.

### Step e) of Pasteurizing

In an embodiment, the method comprises an additional step e) of pasteurizing of the composition (e.g. obtained after step c) or step d)) to obtain a pasteurized composition comprising phycobiliprotein-containing micro-organisms. Preferably, said pasteurizing is carried out at a temperature between 6° and 100° C. for a duration of between 5 seconds and 30 minutes, more preferably at a temperature between 7° and 95° C. for a duration of between 5 and 50 seconds. Pasteurization of the composition comprising phycobiliprotein-containing micro-organism has the advantage that it prolongs shelf life. Since pasteurization not only destroys pathogenic micro-organism, but also the beneficial bacteria, pasteurization is a way to make the high nutritional composition of the invention available to people and animals intolerant to probiotics.

In an embodiment, pectin may be added before drying and/or pasteurization in order to increase the stability of the colour; the amount of pectin may be between 0.1 and 1 wt. % based on the weight of composition. A person skilled in the art will be aware of types of pectins that may be used.

With the present invention even after pasteurizing a composition is obtained that has a stable colour value, in other words the cyanobacteria composition according to the present invention is pasteurizable.

### Step f) of Concentrating or Drying

The main object of the present invention is to provide aqueous composition which have optimal nutritional properties. For certain applications it is desirable to concentrate the aqueous composition. Although not preferred it is also possible to (fully) dry the composition.

In an embodiment, the method comprises an additional step f) of concentrating or drying the composition (e.g. in step c), step d), or step e)) to obtain a dried composition (with dried, also concentrated is meant). The thus obtained dried product was found to be very stable and suitable for use in various food and feed products. Like the aqueous composition obtained with the method of the invention, also the concentrated or dried product that can be obtained with the method of the invention has good taste, flavour and smell, and is highly nutritional, making it suitable for use in a wide range of products. With the present invention even after concentration/drying the pleasant taste and colour of the compositions is maintained and the dried composition may be reconstituted in water to obtain a composition that has very similar taste and colour than prior to drying. The aqueous composition can be concentrated for example by a falling film evaporation or rotary evaporation to increase the concentration.

In an embodiment, step f) is a concentration step carried out at a temperature of between 2° and 50° C. for a duration of between 1 and 8 hours. In an embodiment, step f) is a drying step carried out at a temperature of between 3° and 140° C. for a duration of between 1 and 24 hours.

### Step g) of Purifying

In an embodiment, the composition according to the present invention is purified using techniques available in the art.

### Specific Embodiments of the Present Invention

In an embodiment, the according to the present invention is related to a method of producing a phycobiliprotein-containing micro-organism-based composition, the method comprising the steps of:
a) reacting fresh phycobiliprotein-containing micro-organisms, water and calcium salt, preferably calcium chloride, for a duration of at least 1 hour to provide an aqueous suspension;
b) adding an acidifier to the aqueous suspension of step a) to obtain an acidic aqueous suspension having a pH between 2 and 5; and
c) removing the solids from the acidic aqueous suspension to obtain the phycobiliprotein-containing micro-organism-based composition.

In an embodiment, the according to the present invention is related to a method of producing a phycobiliprotein-containing micro-organism-based composition, the method comprising the steps of:
a) reacting fresh phycobiliprotein-containing micro-organisms, water and calcium salt, preferably calcium chloride, for a duration of at least 1 hour at a pH >5 to provide an aqueous suspension;
b) adding an acidifier to the aqueous suspension of step a) to obtain an acidic aqueous suspension having a pH between 2 and 5; and
c) removing the solids from the acidic aqueous suspension through centrifugation to obtain the phycobiliprotein-containing micro-organism-based composition.

In an embodiment, the according to the present invention is related to a method of producing a phycobiliprotein-containing composition based on Arthrospira platensis, the method comprising the steps of:
a) reacting fresh Arthrospira platensis, water and calcium salt, preferably calcium chloride, for a duration of at least 1 hour to provide an aqueous suspension;
b) adding an acidifier to the aqueous suspension of step a) to obtain an acidic aqueous suspension having a pH between 2 and 5; and
c) removing the solids from the acidic aqueous suspension to obtain the phycobiliprotein-containing Arthrospira platensis-based composition.

### Effects of the Present Invention

As embodied and broadly described herein, the present invention is directed to the surprising finding that with the method according to the invention a composition can be obtained with components obtained from phycobiliprotein-containing micro-organisms that e.g. has a high nutritional/nourishment profile, and is palatable and savoury as well, that is, devoid of the fishy taste and odour present in products in the prior art.

The present invention allows for obtaining composition that comprise phycobiliproteins, such as phycocyanin, having excellent colour stability and with fast extraction of high amounts of proteins. This requires a three-step approach as provided in the claims wherein in a first step a salt is used for breaking the cell walls whereafter the composition is acidified in a second step and wherein the solids are removed in a third step.

With prior art process such colour-stable compositions are not obtained. As discussed above, processes that do not have a step of acidification do not lead to colour-stable compositions. As discussed above there are prior art processes that use buffers during the extraction process to keep the pH constant. That is required for these processes since the colour is not stable at low pH values and thus it is essential to ensure that the pH value does not decrease to a value of below 6 during this process and that is why the buffer is used. When the pH value would decrease to acidic values, the colour would deteriorate in this prior art process. With the present process, phycobiliprotein-containing compositions can be obtained that can be easily integrated in final consumer applications that require pasteurization processes or long shelf lives.

The present invention now allows for a process that provides a phycobiliprotein-containing composition that is stable at acidic pH and has an increased phycobiliprotein extraction. The stepped process according to the invention provides optimal results in view of improved colour, extended durability of the colour as well as an increased protein extraction. Due to the fact that the extraction is very efficient, the present process allows for a short duration of step a) which has the benefit that the phycobiliproteins that are released and exposed for a shorter time to the high-stress (salt) conditions so that there is less denaturation of the protein and less chance of bacterial growth than a process which would require a longer duration.

The present method thus allows the production of a high nutritional, highly nourishing composition, which can be used as or in food stuff, cosmetics, and skin care products. The composition of the invention may be an edible composition, that is safe for consumption and based on edible starting material. Toxic bacteria are not used in the present invention, the present invention is related to non-toxic compositions.

Without being wishing to be bound by a particular theory, the present inventors have found that during the processing of the phycobiliprotein-containing micro-organisms with the method of the invention the components with bad organoleptic properties are removed, these are mostly insoluble components. Experiments showed that these steps dramatically improved overall taste, and smell and therefore palatability of the obtained composition. Further, test panel evaluation showed that the obtained phycobiliprotein-containing micro-organisms-based composition is characterized by a taste, odour, texture and overall palatability that is highly appreciated by the consumer.

In addition, it was found that the method according to the invention allows for the efficient disruption of the cell wall of the phycobiliprotein-containing micro-organisms, thereby allowing release of internal content of the phycobiliprotein-containing micro-organisms in the composition enhancing digestibility, nutritional profile, and bio-assimilation.

Further, it was found that breaking down the phycobiliprotein-containing micro-organism increased overall solubility of these micro-organisms in the composition (allowing also to increase the amount of the micro-organisms used), and, partly related to the enhanced solubility of the composition, improved texture and appearance of the composition. The method according to the invention allows for improved stability of the product, a good shelf life, effectively providing an environmentally friendly alternative to drying of fresh phycobiliprotein-containing micro-organisms as a means to preserve fresh phycobiliprotein-containing micro-organisms. This significantly increases the sustainability of the product.

It was also found that the composition is a healthy alternative “food to go”, a food product that is ready to consume and can easily be transported to the place of consumption. The composition may be consumed at different temperatures without any loss of taste.

The present inventors have inventively observed that, when compared to prior art products, the present method allows to stabilize the colour for a wide range of pH values and temperatures. The present inventors have observed, without wishing to be bound by a particular theory that at conditions of pH<5 and/or temperatures of more than 40° C., preferably more than 70° C., the phycobiliprotein loses its conformation and therewith its functionality, reaching denaturation if conditions are prolonged. The present compositions do not have this set back and are shown to be stable below pH 3.5/high temperature conditions, thereby avoiding denaturation and maintaining all the nutritional benefits, including the antioxidant effect. The stability at low pH and/or high temperature may for example be measured by the colour stability of the composition. This colour stability is known to be indicative of the overall stability and functionality of the phycobiliprotein.

Therefore, overall the inventors have found a new and inventive method for producing a phycobiliprotein-containing micro-organisms comprising composition, which can be used for instance as food stuff, and/or as an ingredient and/or additive in foodstuff, as colour supplements or as dietary supplements, and as cosmetics, and skin care products, and/or as an ingredient and/or additive in cosmetics, and skin care products.

The method according to the invention allows for the efficient disruption of the cell wall of the phycobiliprotein-containing micro-organisms, thereby allowing release of internal content of the phycobiliprotein-containing micro-organisms in the composition enhancing digestibility, nutritional profile, and bio-assimilation. Breaking down the phycobiliprotein-containing micro-organism, further increases overall solubility of (the components of) said micro-organisms in the composition (allowing also to increase the amount of the micro-organisms used), and, partly related to the enhanced solubility of the composition, improved texture and appearance of the composition. The method according to the invention further allows for improved stability of the product, and good shelf life. Prior art processes focus on obtaining phycocyanin from cyanobacteria, the present invention is able to obtain a large array of nutrients from said cyanobacteria, a larger amount (gram nutrients/kilogram cyanobacteria) is obtained by using the inventive method.

### Composition

In several aspects of the present invention, compositions are obtained, being phycobiliprotein-containing micro-organisms-based compositions. Examples of these compositions are an aqueous water soluble extract composition that is obtained after removal of the solids in step c), a dried water soluble extract composition that is obtained from the aqueous water soluble extract composition after drying, an aqueous non-water soluble extract composition that is obtained as the solids that are removed during step c), and a dried non-water soluble extract composition that is observed from the aqueous non-water soluble extract composition after drying. Each of these are discussed below. It should be noted that the present invention also comprises combinations of one or more thereof, such as a combination of an aqueous water soluble extract composition with (part of) an aqueous non-water soluble extract composition or a combination of an dried water soluble extract composition with (part of) an dried non-water soluble extract composition.

As discussed above, step c) comprises separating the acidic aqueous suspension obtained in step b) into an aqueous phycobiliprotein-containing micro-organism-based water soluble extract composition and a phycobiliprotein-containing micro-organism-based non-water soluble extract composition. Both the water soluble extract compositions (aqueous and dried) as the non-water soluble extract compositions (aqueous and dried) have a high nutritional/nourishment profile, are well digestible and absorbable and are high in probiotics, and thus both may be used, neither one is considered waste.

US2020115423A1 relates to aqueous water soluble extract composition comprising phycocyanin from Galdieria sulphuraria. The process of US2020115423A1 differs from the process of the present invention in that it uses mechanical grinding to break up the cells walls instead of a salt and in that it does not include a step of acidification. The process of US2020115423A1 leads to a different composition having a significantly lower amount of phycocyanin that is extracted.

US2021340490A1 relates to aqueous non-water soluble extract composition comprising phycocyanin from Galdieria. The process of US2021340490A1 differs from the process of the present invention in that it does not use a salt, therefore the process of US2021340490A1 leads to a different composition.

Below the different types of compositions according to the present invention are discussed in more detail.

### Aqueous Water Soluble Extract Composition

In an aspect, the present invention relates to an aqueous phycobiliprotein-containing water soluble extract composition, comprising:
between 40 and 98 wt. % of water;
between 0.5 and 10 wt. % of an acidifier, preferably selected from the group consisting of citric acid, lactic acid, malic acid, lemon juice or lime juice;
at most 0.5 wt. % of chlorophyll, preferably at most 0.001 wt. %; and
at least 0.5 wt. % of phycocyanin, preferably at least 1 wt. %, more preferably at least 10 wt. %, most preferably at least 25 wt. %;
preferably less than 0.1 wt. %, more preferably less than 0.01 wt. % of a sugar,
preferably at least 0.03 wt. %, more preferably at least 0.1 wt. %. of an alkaline or alkaline earth metal salt cation, preferably a calcium cation; and

wherein the composition preferably has:
a colour value according to the CIELAB colour scale for a of between −10 and −4 and for b of between 10 and −40; and/or
a pH of between 2 and 5.

The sum of all these components adds up to 100 wt. % and each of these weight percentages cited are on basis of the total weight of the aqueous water soluble extract composition. In an embodiment, the pH of this aqueous water soluble extract composition is between pH 2 and 5. This pH value corresponds to the pH value during step b) of the process. The aqueous water soluble extract composition according to the present invention may be used as such or may subjected to one or more additional steps as shown above.

### Dried Water Soluble Extract Composition

In an aspect, the present invention relates to a dried phycobiliprotein-containing water soluble extract composition, comprising
between 20 and 60 wt. % of proteins;
between 1 and 20 wt. % of carbohydrates;
between 1 and 50 wt. % of one or more acids;
between 0.5 and 10 wt. % of fat;
at most 1 wt. % of chlorophyll, preferably at most 0.1 wt. %; and
at least 5 wt. % of phycocyanin, such as at least 25 wt. % of phycocyanin, preferably at least 40 wt. %, more preferably at least 50 wt. %;
preferably at least 1.5 wt. %, more preferably at most 5 wt. % of an alkaline or alkaline earth metal salt cation, preferably a calcium cation;
preferably less than 1 wt. %, more preferably less than 0.1 wt. % of a sugar; and

wherein the composition preferably has a pH of between 2 and 5.

The sum of all these components adds up to 100 wt. % and each of these weight percentages cited are on basis of the total weight of the dried water soluble extract composition.

In an embodiment, the pH of this aqueous water soluble extract composition is between pH 2 and 5. This pH value corresponds to the pH value during step b) of the process. The dried water soluble extract composition according to the present invention may be used as such or may subjected to one or more additional steps as shown above.

### Aqueous Non-Water Soluble Extract Composition

In an aspect, the present invention relates to an aqueous phycobiliprotein-containing non-water soluble extract composition comprising:
between 50 and 98 wt. % of water;
between 1 and 50 wt. % of protein;
between 1 and 50 wt. % of an acidifier, preferably citric acid, lactic acid, malic acid, lemon juice or lime juice;
at least 0.01 wt. % of chlorophyll, such as at least 0.5 wt. %, preferably at least 1 wt. %, such as at least 2 wt. %;
at least 0.1 wt. % of phycocyanin;
preferably at least 0.03 wt. %, more preferably at least 0.1 wt. % of an alkaline or alkaline earth metal salt cation, preferably a calcium cation; and

wherein the composition preferably has a pH of between 2 and 5.

The sum of all these components adds up to 100 wt. % and each of these weight percentages cited are on basis of the total weight of the aqueous non-water soluble extract composition.

In an embodiment, the pH of this aqueous water soluble extract composition is between pH 2 and 5. This pH value corresponds to the pH value during step b) of the process. The aqueous non-water soluble extract composition according to the present invention may be used as such or may subjected to one or more additional steps as shown above.

### Dried Non-Water Soluble Extract Composition

In an aspect, the present invention relates to a dried phycobiliprotein-containing non-water soluble extract composition comprising:
between 10 and 90 wt. % of proteins;
between 1 and 40 wt. % of carbohydrates;
between 1 and 50 wt. % of an acid
between 1 and 40 wt. % of fat;
at least 1 wt. % of chlorophyll, preferably at least 2 wt. %; such as at least 4 wt. %;
at most 10 wt. % of phycocyanin, preferably at most 0.1 wt. %.
preferably at least 1.5 wt. %, more preferably at least 5 wt. %. of an alkaline or alkaline earth metal salt cation, preferably a calcium cation; and

wherein the composition preferably has a pH of between 2 and 5.

The sum of all these components adds up to 100 wt. % and each of these weight percentages cited are on basis of the total weight of the dried non-water soluble extract composition.

In an embodiment, the pH of this aqueous water soluble extract composition is between pH 2 and 5. This pH value corresponds to the pH value during step b) of the process. The dried non-water soluble extract composition according to the present invention may be used as such or may subjected to one or more additional steps as shown above.

### Colour Value

In an embodiment, said aqueous water soluble extract composition obtained has a colour value according to the CIELAB Colour scale for a of between −10 and −4 and for b of between 10 and −40 at dry weight of the aqueous water soluble extract composition of between 0.5 and 6 wt. %. With higher levels of solid matter (higher dry weight), viz. concentration, the CIELAB values will differ. The CIELAB Colour values were determined using a Hunterlab Ultrascan VIS. Without wishing to be bound by a particular theory, the present inventors believe that by using the present process, the colour stability is increased. In addition, the phycocyanin is not isolated from its natural environment and by keeping other cell components, the colour stability of the phycocyanin is increased. This is an advantage obtained by the method according to the present invention.

The colour value is stable even at temperature of pasteurization. In an embodiment, the colour is stable at pH between 8 and 2.5.

In an embodiment, said dried water soluble extract composition is prepared from an aqueous water soluble extract composition having a colour value as discussed for the aspect of the method above. The colour value may be stable for at least 4 months, preferably at least 12 months.

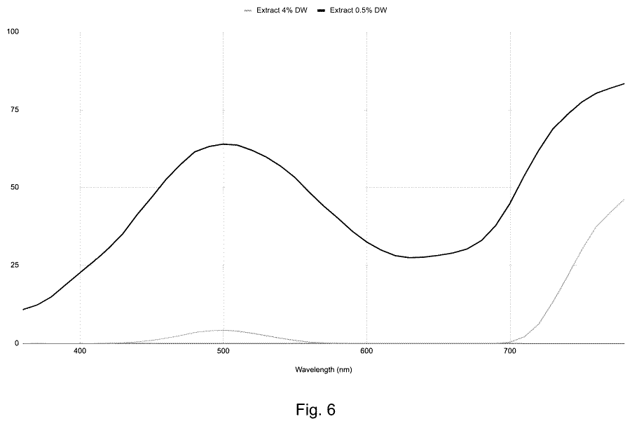

In an embodiment, the aqueous water soluble extract composition has peak transmittance factors at a wavelength of >700 nanometre and between 450 and 550 nanometre. This is also visible in FIG. 6. The transmittance factors are measured using a Hunterlab Ultrascan VIS.

### Use of Compositions

The composition (aqueous and/or dried and water soluble extract and/or non-water soluble extract) according to the present invention may be used as drink or in drinks, in food and feed.

In an embodiment in which lactic acid fermentation is used, the composition obtained by the method of the present invention provides a dairy-free alternative for lacto-fermented products from milk. The demand for dairy-free alternatives is rising due to the increasing incidence of lactose intolerance and veganism, and as a response to the request for higher nutritional and nourishing quality and fortified food products, cosmetics and skin care products. Phycobiliprotein-containing micro-organisms represent a suitable substrate for the production of fermented foods due to their availability and high nutritional value. In addition, since the number of lactic acid bacteria increases during fermentation, the composition obtained through the method of the invention, contains probiotics providing health benefits, in particular by restoring and enhancing the composition of the gut microbiome.

### EXAMPLES

The present invention is further elucidated based on the Examples below which are illustrative only and not considered limiting to the present invention.

### Example 1: Effect of Amount of Salt

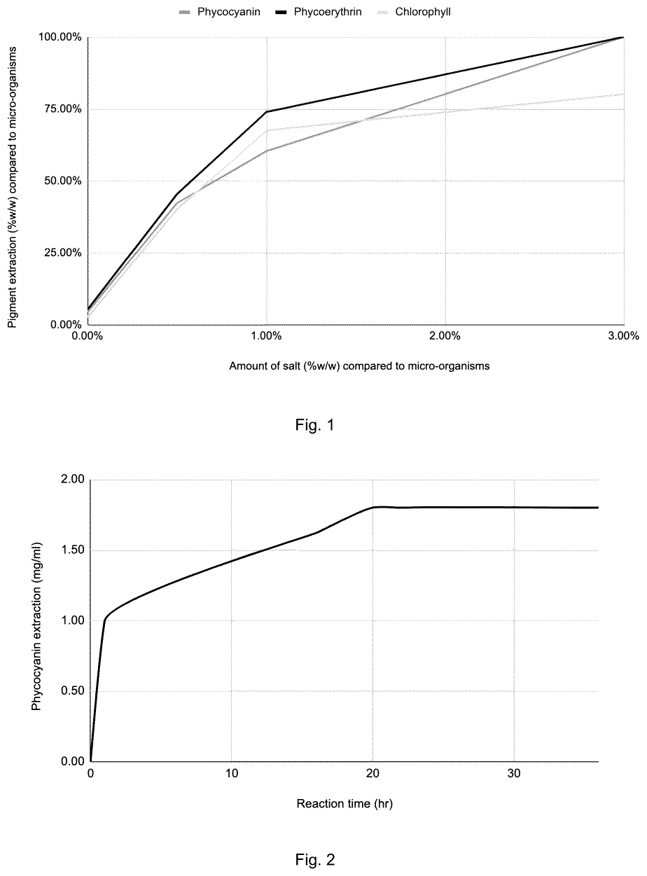

The effect of the amount of salt has been tested in this example. Salt was shown to have an effect on the recovery of pigments present in phycobiliprotein-containing micro-organisms, being chlorophyll, phycocyanin, and phycoerythrin. The amount of salt compared to the amount of phycobiliprotein-containing micro-organisms used is varied and the recovered amount of phycocyanin (dark grey curve), phycoerythrin (black curve) and chlorophyll (light grey curve) curve is shown in FIG. 1.

Table 1 below shows the amount of recovered pigments from the phycobiliprotein-containing micro-organisms. As microorganism Arthrospira Platensis is used, this microorganism is added in an amount of 25 gram to 100 ml of solution of demineralized water and different concentrations of CaCl₂ and kept at a temperature of 5° C. for a duration of 16 hours with continuous agitation. After that time, samples were centrifuged during 10 minutes at relative centrifugal force (RCF) of 10000 using a MIKRO 22 Hettich centrifuge and the supernatant separated to obtain the aqueous water soluble extract composition according to the present invention.

Absorbances of the aqueous composition at wavelengths 620, 652, 565 and 465 nm were measured using SmartSpec™ Plus spectrophotometer. In the case of phycocyanin, the absorbances at 620 nm and 652 nm are used to calculate the amount of phycocyanin using the Moon et al., 2013 publication (Isolation and characterization of thermostable phycocyanin from Galdieria sulphuraria. Myounghoon Moon, Sanjiv K. Mishra, Chul Woong Kim, William I. Suh, Min S. Park*Min S. Park, and Ji-Won Yang) according to the following equation:

$$Phycocyanin ( mg {mL}^{- 1} ) = [ {A}_{620} - 0.474 ( {A}_{652} ) ] / 5.34$$

In case of chlorophyll and phycoerythrin, absorbance at 465 nm for chlorophyll A and absorbance at 565 nm for phycoerythrin and are used as a proxy.

With percentage recovery is meant the amount that is recovered in the aqueous water soluble extract composition as a fraction compared to the amount present in the phycobiliprotein-containing micro-organisms that were used as starting material (information given from supplier).

TABLE 1

| CaCl2 in view of | | | |
|---|---|---|---|
| phycobiliprotein-containing | phycocyanin | phycoerythrin | chlorophyll |
| micro-organisms (% w/w) | % recovery | % recovery | % recovery |
| 0.0 | 4.376 | 5.317 | 2,578 |
| 0.5 | 42.33 | 45.45 | 40.24 |
| 1 | 60.34 | 73.93 | 67.65 |
| 2 | 97.03 | 98.33 | 75.17 |
| 3 | 100.0 | 100.0 | 80.22 |

From this data it is clear that the addition of a salt was found to improve the recovery of pigments. The lower limit to achieve a recovery of above 50% is 2.5%.

### Example 2: Effect of Duration of Step a)

The effect of duration of step a) was tested. As micro-organism Arthrospira Platensis is used, this micro-organism is added in an amount of 25 gram to 100 ml of solution of demineralized water and different concentrations of CaCl2 and kept at a temperature of 5° C. for a duration of 16 hours with continuous agitation. After that time, samples were centrifuged during 10 minutes at relative centrifugal force of 10000 using a MIKRO 22 Hettich centrifuge and the supernatant separated as the aqueous water soluble extract composition.

Absorbances of the aqueous water soluble extract composition at wavelengths 620 and 652 nm were measured using SmartSpec™ Plus spectrophotometer. In the case of phycocyanin, the absorbances at 620 nm and 652 nm are used to calculate the amount of phycocyanin using the using the Moon et al., 2013 publication as shown above.

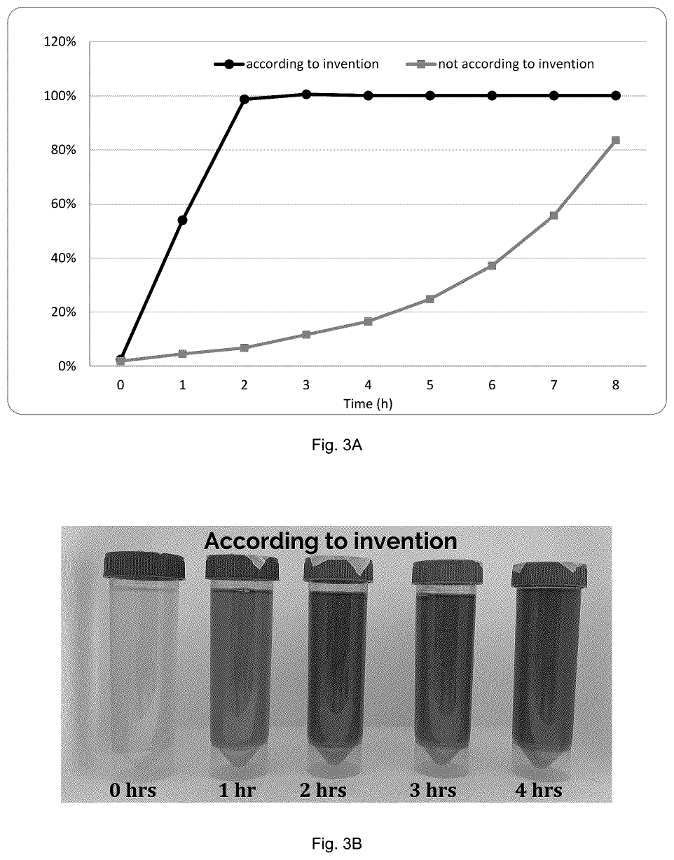

FIG. 2 shows a graph in which the amount of phycocyanin (in mg/ml) in plotted in relation to the duration of step a. From FIG. 2 is clear that at a duration of 1 hour for step a), there is already effect but the effect increases with increasing duration.

### Example 3: Effect of Using a Buffer Instead of a Salt

The effect of using a salt versus the use of a buffer is tested in this Example. As micro-organism Arthrospira Platensis is used. For the test according to the invention an amount of 25 gram of Arthrospira Platensis in fresh form was added to 100 ml of a solution of 1.2 wt. % of CaCl₂ in demineralized water at a temperature of 5° C., the pH was 6. The mixture was kept at that temperature and pH for different durations. For the test not according to the invention (comparable to CN113278528A) an amount of 25 gram of Arthrospira Platensis in fresh form was added to 100 ml of a 10 mM citric acid-sodium citrate buffer solution (prepared with 10 mM citric acid solution and 10 mM sodium citrate solution both in demineralized water) at a temperature of 5° C., the pH was 6. The mixture was kept at that temperature and pH for different durations.

For both tests, the mixtures were centrifuged during 10 minutes at relative centrifugal force (RCF) of 10000 using a MIKRO 22 Hettich centrifuge. The supernatant is separated as the aqueous water soluble extract composition.

Absorbances of the aqueous water soluble extract compositions at wavelengths 620 and 652 nm were measured using SmartSpec™ Plus spectrophotometer. In the case of phycocyanin, the absorbances at 620 nm and 652 nm are used to calculate the amount of phycocyanin using the using the Moon et al., 2013 publication as shown 20 above.

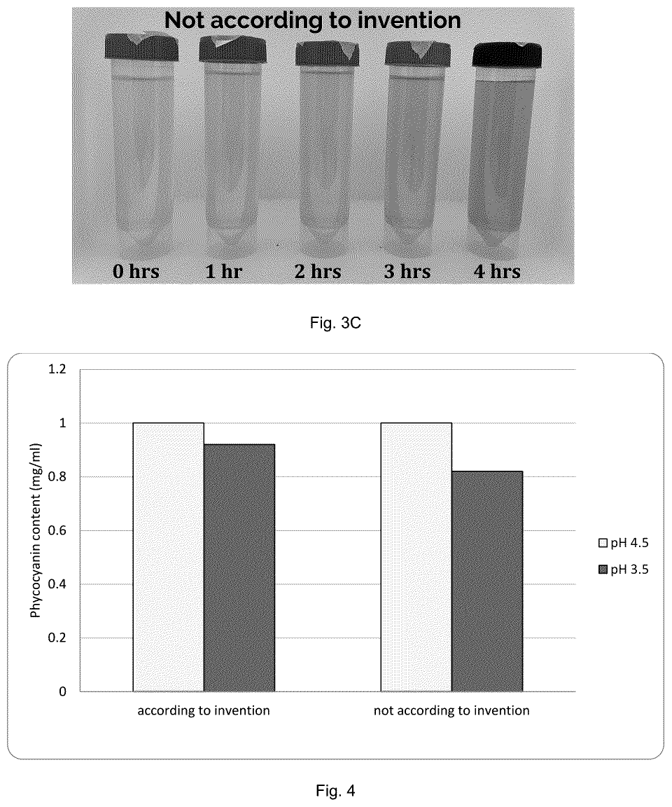

FIG. 3A shows a graph in which the recovery amount of phycocyanin (in mg/ml) in plotted in relation to the duration of the extraction step. The curve for the test according to the invention (using alkaline salt) is provided in black with circles and the curve for the test not according to the invention (using a buffer) is provided in grey with grey squares. From the graph in FIG. 3A it is very clear that with the method according to the invention (using a salt), after 1 hour the extraction was over 50% whereas with the method not according to the invention (using a buffer) the extraction was only 5%. After 2 hours, the method according to the invention led to 99% whereas with the method not according to the invention the extraction was only 7%. It should be noted that even after 8 hours, the extraction using the buffer was in this experiment no higher than 83.5%.

FIGS. 3B and 3C shows pictures in which is clearly visible that the blue colour (presence of phycocyanin) is present stronger and earlier in the test according to the invention (FIG. 3B) compared to the test not according to the invention (FIG. 3C). Moreover, FIG. 3B shows that after 2 hours, the colour is already very strong.

When visually observing the samples, it was found that the samples according to the invention are blue wherein the samples not according to the invention are more greenish-blue, leading to the conclusion that this prior art method is more efficient at extracting green components than the blue phycocyanin components.

### Example 4: Effect of Using an Acidic pH in Step b)

In this example, the effect of using an acidic pH in step b) was tested. Two tests were carried out, one according to the invention with a step a) using a salt and a step b) using an acidic pH by adding an acidifier and one not according to the invention with a step a) using a salt and a step b) without using an acidic pH.

As micro-organism Arthrospira Platensis is used. For both the test according to the invention and the test not according to the invention, both an amount of 25 gram of Arthrospira Platensis in fresh form was added to 100 ml of a solution of 1.5 wt. % of CaCl₂ in demineralized water at a temperature of 5° C., the pH was 6. The mixture was kept at that temperature and pH for a duration of 8 hours.

For the test according to the invention the composition was acidified using citric acid to a pH value of 4.5. For the test not according to the invention, no acidification was carried out and the pH remaining 6.

For both tests, the mixtures were centrifuged during 10 minutes at relative centrifugal force (RCF) of 10000 using a MIKRO 22 Hettich centrifuge. The supernatant is separated as the aqueous water soluble extract composition.

For the first sample according to the invention, the pH is 4.5 and is retained.

For the first sample not according to the invention, the pH was brought to pH by addition of the appropriate amount of citric acid.

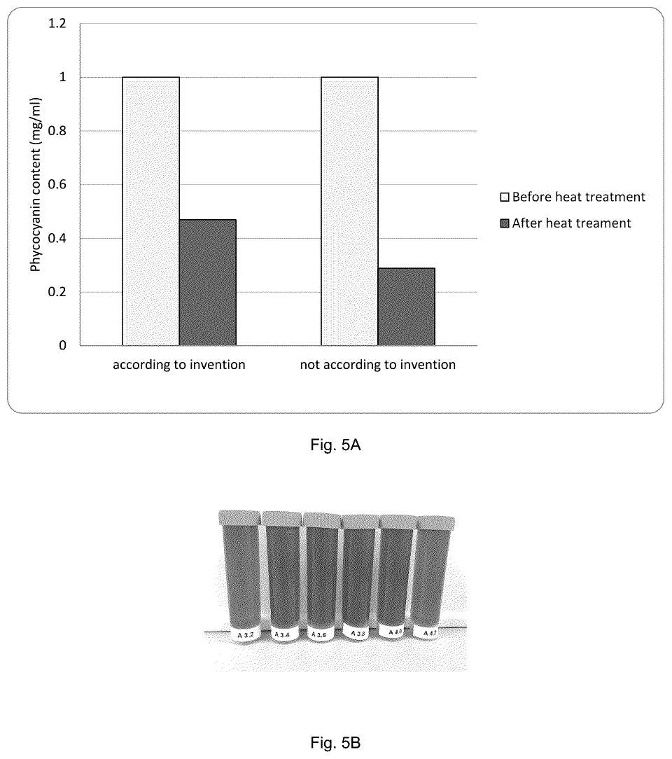

For both of these samples, the pH is the same, namely 4.5. The samples were diluted to obtain a final concentrations of 1 mg/mL of phycocyanin. Of these samples absorbances at 620 nm and 652 nm are measured and used to calculate the amount of phycocyanin using the using the Moon et al., 2013 publication as shown above. The results are provided in the graph of FIG. 4 as the light grey column that have a set phycocyanin content of 1 mg/mL. These are used as the “100%” value to see the effect of when the pH is further lowered to pH 3.5.

Both the sample according and not according to the invention are brought from pH 4.5 to pH 3.5 by addition of the appropriate amount of citric acid. The absorbances at 620 nm and 652 nm are measured and used to calculate the amount of phycocyanin using the using the Moon et al., 2013 publication as shown above. FIG. 4 shows in dark grey the columns with the content of phycocyanin (in mg/ml) that measured for these samples, after the drop in pH.

The results clearly show that for the sample not according to the invention, there was significant loss of colour of 18% when going from pH 4.5 to pH 3.5, wherein with the sample according to the present invention, that loss was only 8%. This clearly shows that the acidic pH during step b) has an effect of stabilizing the colour at a low pH.

### Example 5: Effect of Heat Treatment on the Colour Value

### Example 5A: Effect of Heat Treatment on Samples of Example 4

In this example, the effect of heat treatment on samples that were prepared according to the invention (using acidic pH during step b) and not according to the invention (not using acidic pH during step b) were tested. Two tests were carried out, one according to the invention with a step a) using a salt and a step b) using an acidic pH by adding an acidifier and one not according to the invention with a step a) using a salt and a step b) without using an acidic pH.

As micro-organism Arthrospira Platensis is used. For both the test according to the invention and the test not according to the invention, both an amount of 25 gram of Arthrospira Platensis in fresh form was added to 100 ml of a solution of 1.5 wt. % of CaCl₂ in demineralized water at a temperature of 5° C., the pH was 6. The mixture was kept at that temperature and pH for a duration of 8 hours. This is the same as in Example 4.

For the test according to the invention the composition was acidified using citric acid to a pH value of 4.5. For the test not according to the invention, no acidification was carried out and the pH remained at pH 6.

For both tests, the mixtures were centrifuged during 10 minutes at relative centrifugal force (RCF) of 10000 using a MIKRO 22 Hettich centrifuge. The supernatant is separated as the aqueous water soluble extract composition.

For both tests, the aqueous water soluble extract composition obtained were diluted to a final concentration of 1 mg/mL of phycocyanin to have the same starting amount to clearly show the effect of heat treatment only. The samples were then heat treated at a temperature of 70° C. for a period of 5 minutes. Both samples were left to cool down at room temperature for 1 hour.

The absorbances at 620 nm and 652 nm are measured and used to calculate the amount of phycocyanin using the using the Moon et al., 2013 publication as shown above.

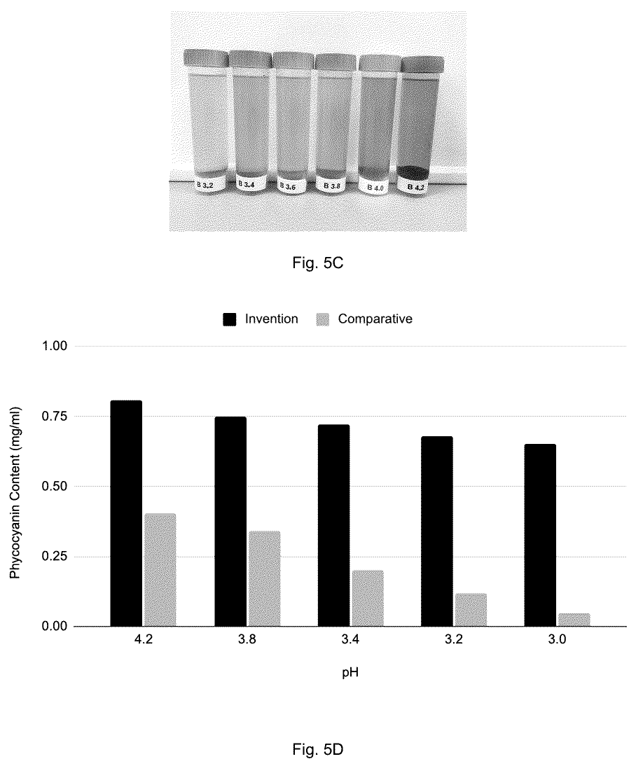

FIG. 5A shows a graph in which the content of phycocyanin (in mg/ml) is measured for each samples before and after heat treatment. The results clearly show that for the test not according to the invention, there was significant loss of colour, since only 30% of the extracted phycocyanin remained after heat treatment. In the example according to the invention close to 50% of the phycocyanin content was retained after intense heat treatment.

This clearly shows that the with the process according to the invention, the colour stability is increased by a factor of two. This improvement is due to better stability of the extract when it is separated at a pH between 2-5, which ensures natural preservation of phycocyanin bonds.

### Example 5B: Effect of Pasteurization

The present inventors have tested the effect of pasteurization at elevated temperature on the stability of the colour value for compositions according to the present invention and comparative compositions. This example clearly shows that with the present method composition can be obtained with superior colour value stability at high temperature over a broad range of pH.

As microorganism Arthrospira Platensis is used, this microorganism is added in an amount of 25 gram to 100 ml of solution of demineralized water and 4% solution of CaCl2 and kept at a temperature of 5° C. for a duration of 16 hours with continuous agitation. The pH of the mix was lowered to 4.5. The resulting mix was centrifuged during 10 minutes at RFC 10000 (MIKRO 22 Hettich). The supernatant was separated through decantation. The resulting mix was diluted until the final phycocyanin concentration was 0.5%.

As a comparative phycocyanin in powder form (commercially available form e.g. Mattison) was tested for stability by mixing 1 gram in 100 millilitre of demineralized water at a temperature of 10° C. The resulting mix was diluted until the final phycocyanin concentration was 0.5%. The concentration of phycocyanin in the final mix was determined using the method and equation shown above.

Both compositions were brought to the desired pH levels of pH 3.0, pH 3.2, pH 3.4, pH 3.8, and pH 4.2 by addition of the appropriate amount of citric acid. The resulting mixtures were pasteurized at 85° C. during 10 min. The resulting compositions were tested visually for their colour value and stability against sedimentation. The black and white photographs shown in FIG. 5B for the compositions according to the present invention and in FIG. 5C for the composition according to the prior art. FIG. 5D shows the concentration of phycocyanin for the aqueous water soluble extract composition according to the present invention (black columns) and the prior art compositions (grey columns), clearly showing that the compositions according to the present invention have a significantly higher amount of phycocyanin at all pH levels and in addition showing that the stability at low pH is higher for the present compositions than for the prior art compositions. The prior art compositions show poor stability at low pH and form sediment and become turbid. This example shows the excellent colour stability of the present compositions at low pH.

### Example 6: Colour Value

An aqueous water soluble extract composition was prepared as discussed in Example 3. Part of this composition was diluted to a dry weight amount of 4% and part of this composition was diluted to a dry weight amount of 0.5%. Both of these were adjusted to a pH of 4.5. Then the transmittance was measured using a Hunterlab Ultrascan VIS providing the curves shown in FIG. 4, being a black curve for a composition having a pH of 4.5 and 4 wt. % of dry matter and a grey curve for a composition having a pH of 4.5 and 0.5 wt. % of dry matter. These curves clearly show that both curves have a first peak transmittance factor (highest peak) at a wavelength of >700 nanometre and a second peak transmittance factor (second highest peak) of approx. 500 nanometre.

The scope of the present invention is defined by the appended claims. One or more of the objects of the invention are achieved by the appended claims.

1. A method of producing a phycobiliprotein-containing micro-organism-based composition, the method comprising the steps of.
a) reacting phycobiliprotein-containing micro-organisms, water and a salt for a duration of at least 1 hour to provide an aqueous suspension;
b) obtaining an acidic aqueous suspension having a pH between 2 and 7 starting from the aqueous suspension obtained in step a);
c) removing the solids from the acidic aqueous suspension to obtain the phycobiliprotein-containing micro-organism-based composition.

2. The method according to claim 1, wherein in step a) the salt is an alkaline or alkaline earth metal salt, and wherein step b) comprises adding an acidifier to the aqueous suspension of step a) to obtain the acidic aqueous suspension having a pH between 2 and 5.

3. The method according to claim 1, wherein step b) comprises the steps of.
b-I) adding lactic acid bacteria to the aqueous suspension obtained in step a) to obtain an aqueous suspension with lactic acid bacteria;
b-II) allowing the aqueous suspension with lactic acid bacteria to ferment until a pH between 2 and 5 is obtained;
to obtain the acidic aqueous suspension having a pH between 2 and 5.

4. The method according to claim 1, wherein as phycobiliprotein-containing micro-organisms cyanobacteria are used to prepare a cyanobacteria-based composition.

5. The method according to claim 1, wherein fresh phycobiliprotein-containing micro-organisms are used.

6. The method according to claim 1, comprising one or more additional steps:
an additional step d) of washing the solids removed in step c) to obtain additional phycobiliprotein-containing micro-organism-based composition that is combined with the phycobiliprotein-containing micro-organism-based composition obtained in step c) and/or
an additional step e) of pasteurizing of the phycobiliprotein-containing micro-organism-based composition obtained in step c) and/or step d) to obtain a pasteurized phycobiliprotein-containing micro-organism-based composition, and/or
an additional step f) of concentrating or drying the phycobiliprotein-containing micro-organism-based composition obtained in step c) and/or step e) or the pasteurized phycobiliprotein-containing micro-organism-based composition obtained in step e) to obtain a concentrated or dried (pasteurized) phycobiliprotein-containing micro-organism-based composition.

7. The method according to claim 1, wherein step c) comprises separating the acidic aqueous suspension obtained in step b) into an aqueous water soluble extract composition and a non-water soluble extract composition.

8. The method according to claim 1, wherein in step c) is carried out using centrifugation.

9. The method according to claim 1, wherein said phycobiliprotein-containing micro-organism-based composition obtained has a colour value according to the CIELAB Colour scale for a of between −10 and −4 and for b of between 10 and −40.

10. The method according to claim 1, wherein during step a) the temperature is at most 5° C. and the pH is >5.

11. An aqueous phycobiliprotein-containing water soluble extract composition, comprising:
between 40 and 98 wt. % of water;
between 0.5 and 10 wt. % of an acidifier;
at most 0.5 wt. % of chlorophyll; and
at least 0.5 wt. % of phycocyanin;

12. A dried phycobiliprotein-containing water soluble extract composition, comprising:
between 20 and 60 wt. % of proteins;
between 1 and 20 wt. % of carbohydrates;
between 1 and 50 wt. % of one or more acids;
between 0.5 and 10 wt. % of fat;
at most 1 wt. % of chlorophyll; and
at least 5 wt. % of phycocyanin;

13. An aqueous phycobiliprotein-containing non-water soluble extract composition comprising:
between 50 and 98 wt. % of water;
between 1 and 50 wt. % of protein;
between 1 and 50 wt. % of an acidifier;
at least 0.01 wt. % of chlorophyll;
at least 0.1 wt. % of phycocyanin.

14. A dried phycobiliprotein-containing non-water soluble extract composition comprising:
between 10 and 90 wt. % of proteins;
between 1 and 40 wt. % of carbohydrates;
between 1 and 50 wt. % of an acid
between 1 and 40 wt. % of fat;
at least 1 wt. % of chlorophyll;
at most 10 wt. % of phycocyanin.

15. The method according to claim 6, wherein pasteurizing is carried out at a temperature between 6° and 100° C. for a duration of between 5 seconds and 30 minutes.

16. The aqueous phycobiliprotein-containing water soluble extract composition according to claim 11, wherein the composition has:
a colour value according to the CIELAB colour scale for a of between −10 and −4 and for b of between 10 and −40.

17. The aqueous phycobiliprotein-containing water soluble extract composition according to claim 11, wherein the composition has a pH of between 2 and 5.

18. The dried phycobiliprotein-containing water soluble extract composition according to claim 12, wherein the composition has a pH of between 2 and 5.

19. The aqueous phycobiliprotein-containing non-water soluble extract composition according to claim 13, wherein the composition has a pH of between 2 and 5

20. The dried phycobiliprotein-containing non-water soluble extract composition according to claim 14, wherein the composition has a pH of between 2 and 5.
